(19) United States
(12) Patent Application Publication
(10) Pub. No.: US 2004/0254530 A1
(43) Pub. Date: Dec. 16, 2004

(54) Destructible safety syringe and barrel thereof

(76) Inventors: Shu-Chen Chou, Taipei (TW); Chao-Hsing Cheng, Taipei (TW)

Correspondence Address: BIRCH STEWART KOLASCH & BIRCH, PO BOX 747, FALLS CHURCH, VA, 22040-0747, US

(21) Appl. No.: 10/692,788

(22) Filed: Oct. 27, 2003

(30) Foreign Application Priority Data
Jun. 16, 2003 (TW) 92116242

(51) Int. Cl. A61M 5/00

(52) U.S. Cl. 604/110

(57) ABSTRACT

A destructible safety syringe and a barrel thereof are provided. The destructible safety syringe includes a needle hub, a needle seat, a body, a destruction device, a cover and a plunger. The needle seat is connected to a first end of the body by a connection portion. The needle seat and the body are made by injection molding method. The destruction device and the cover are movably connected to the body. After use of the syringe or the barrel, the destruction device is rotated to break the connection portion to destructively separate the needle seat from the body. The cover is provided to shelter the first end of the body, and then the plunger is pushed to damage the needle hub.

### CROSS REFERENCE TO RELATED APPLICATIONS

This application claims priority of Taiwan Patent Application Serial No. 92116242 entitled “Destructible Safety Syringe and Barrel Thereof”, filed on Jun. 16, 2003.

### FIELD OF THE INVENTION

The present invention relates to a destructible safety syringe and a barrel thereof, and particularly to a safety syringe includes a needle seat and a body molded as an integral one-piece casting, wherein the needle seat is destructively separated from the body by a destruction device after use.

### BACKGROUND OF THE INVENTION

FIG. 1 shows a cross-sectional view of a conventional syringe 1. The syringe 1 includes a body 12, a needle seat 14, a needle hub 16, and a plunger 18. The needle hub 16 is removed after use.

FIG. 2 shows a cross-sectional view of another conventional safety syringe 2. The safety syringe 2 includes a body 22, a needle seat 24, an elastic ring 26, a needle hub 16, and a plunger 28. After use of the safety syringe 2, the plunger 28 is engaged with the needle seat 24, and then the needle seat 24 and the needle hub 16 are together pulled into the body 22. In order to pull the needle seat 24, the body 22 and the needle seat 24 are individually molded, and so fluid may leak out from between the needle seat 24 and the body 22. Therefore, an additional elastic ring 26 is disposed on the needle seat 24 and hermetically contacts with the inner surface of the body 22. Some modifications with similar function of the elastic ring 26 are also provided in other conventional safety syringes, but they are not elaborated here for conciseness.

There are some drawbacks of the above-mentioned syringes. First, the needle seat and the body are not thoroughly broken and can not prevent inappropriate recycling or reuse. Second, the additional anti-leakage design complicates the production of the syringe. Last, the needle hub still may stick out of the body and cause injuries when an external force accidentally pushes the plunger of the pullback-type safety syringe.

### SUMMARY OF THE INVENTION

One aspect of the present invention is to provide a destructible safety syringe and a barrel thereof for preventing inappropriate recycling and reuse.

Another aspect of the present invention is to provide a destructible safety syringe and a barrel thereof, wherein the needle seat and the body are molded as an integral one-piece casting to simplify the production of the syringe.

Still another aspect of the present invention is to provide a destructible safety syringe and barrel thereof, wherein a destruction device is movably disposed on the body to destructively separate the needle seat from the body.

Still another aspect of the present invention is to provide a destructible safety syringe and barrel thereof, wherein a cover is movably disposed on one end of the body and shelters the end of the body after use to prevent the needle hub from accidentally hurting people. The needle hub is further broken by pushing the plunger and compresses the needle seat.

One embodiment of the present invention discloses a destructible barrel of a safety syringe and the destructible barrel includes a body, a needle seat, a destruction device, and a cover. The body contains an injection fluid, and has a first end and a second end. The needle seat is connected to the first end of the body through a connection portion, and supports a needle hub. The destruction device is movably engaged with the first end of the body. The cover is movably connected to the destruction device, and includes an opening part and a closed part. The opening part of the cover exposes the first end of the body, and the needle seat passes the cover through the opening part. The needle seat comprises a first spiral surface and the destruction device comprises a second spiral surface corresponding to the first spiral surface. It should be noted that although the spiral surface is used as an exemplary embodiment here, the scope of the present invention is not limited thereto. Some modifications are known to those skilled in the art, such as a sloping surface or a curved surface.

After use of the destructible barrel, the destruction device is rotated. During rotating the destruction device, the second spiral surface pushes against the first spiral surface along the axis of the barrel due to the height difference between the first and the second spiral surfaces. Furthermore, because the strength of the connection portion is weaker than the strength of the needle seat, the connection portion is broken when the needle seat is pushed. Thus, the needle seat is destructively separated from the body. At last, the cover is moved to shelter the first end of the body, and the needle hub and the needle seat are accommodated inside the body of the barrel.

Another embodiment of the present invention discloses a destructible safety syringe including a body, a needle hub, a needle seat, a destruction device, a cover, and a plunger. The body contains an injection fluid, and the body has a first end and a second end. The needle seat is connected to the first end of the body through a connection portion, and supports the needle hub. The plunger is disposed inside the body and is able to move along the axis for driving the injection fluid. The destruction device is movably engaged with the first end of the body. The body and the needle seat are molded as an integral one-piece casting. The cover is movably connected to the destruction device and includes an opening part and a closed part. The opening part of the cover exposes the first end of the body, and the needle seat passes the cover through the opening part.

The destruction process of this embodiment is similar to that of the above-mentioned embodiment, and is not elaborated here for conciseness. After the destruction of the needle seat, the needle seat is destructively separated from the body. Further, the plunger is pulled to the second end of the body, and an accommodation space inside the body is formed. The cover is pushed to shelter the first end of the body, and the needle hub and the needle seat are accommodated in the body. At last, the plunger is pushed to compress the accommodation space. Thus, the needle hub is deformed and broken.

### BRIEF DESCRIPTION OF THE DRAWINGS

FIG. 1 is a cross-sectional view of a conventional syringe;

FIG. 2 is a cross-sectional view of a conventional safety syringe;

FIG. 3 is a cross-sectional view of a destructible barrel in accordance with one embodiment of the present invention;

FIG. 4 is an explosive view of the destructible barrel in FIG. 3;

FIG. 5A is a perspective view illustrating the body, the needle seat, and the destruction device;

FIG. 5B is a cross-sectional view illustrating the body, the needle seat, and the destruction device;

FIG. 6A is a cross-sectional view of the destructible barrel illustrating the needle seat separated from the body;

FIG. 6B is a perspective view of the destructible barrel illustrating the needle seat separated from the body;

FIG. 7 illustrates the cover sheltering the first end of the body;

FIG. 8 is an explosive view of the destructible barrel in accordance with another embodiment of the present invention; and

FIG. 9 illustrates the breaking process of the needle hub of the syringe.

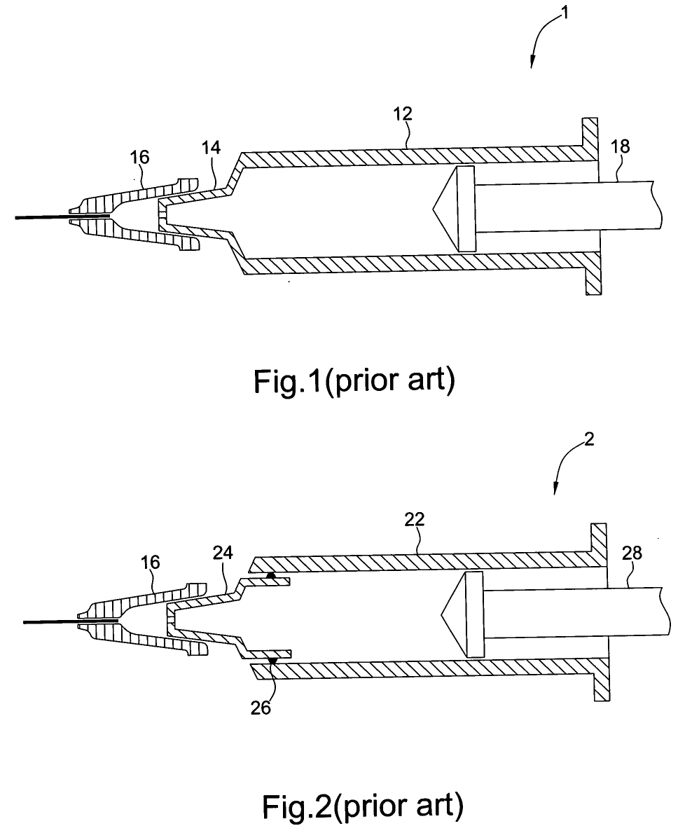

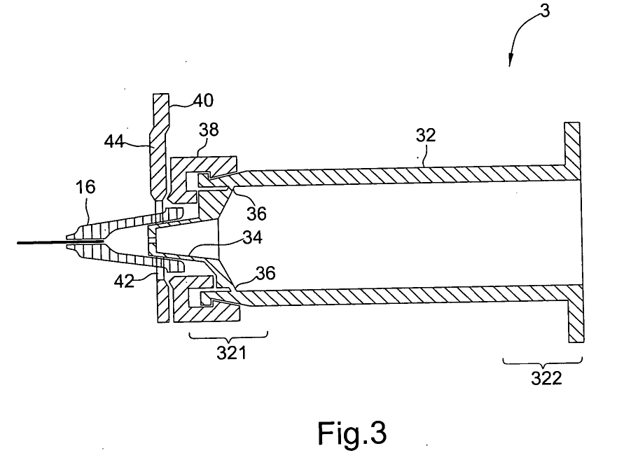

### DETAILED DESCRIPTION OF THE INVENTION

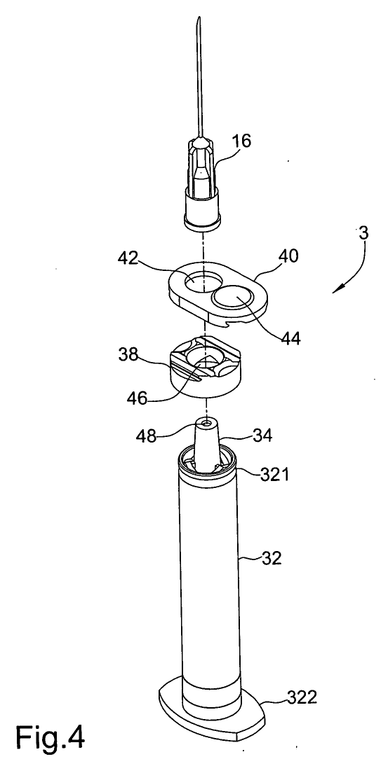

FIG. 3 is a cross-sectional view of a destructible barrel 3 in accordance with one embodiment of the present invention, and the barrel 3 is used in a syringe. The destructible barrel 3 includes a body 32, a needle seat 34, a destruction device 38 and a cover 40. The body 32 contains an injection fluid (not illustrated). The body 32 has a first end 321 and a second end 322. The needle seat 34 is connected to the body 32 through a connection portion 36. The destruction device 38 is movably connected to the first end 321 of the body 32. The body 32 and the needle seat 34 are molded as an integral one-piece casting, and can be made by any well-known method, such as injection molding. Thus, not only the production method is simplified, but the production cost is lowered. Furthermore, the one-piece casting prevents the injection fluid from leaking, and so an additional elastic ring between the body 32 and the needle seat 34 is not required. The cover 40 has an opening part 42 and a closed part 44. The cover 40 is movably connected to the destruction device 38. The opening part 42 exposes the first end 321 of the body 32, and the needle seat 34 passes the cover 40 through the opening part 42.

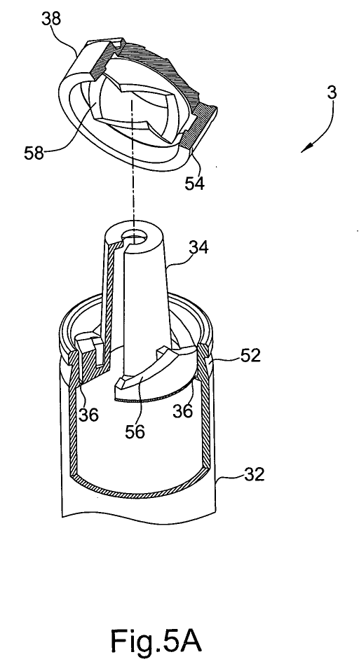

FIG. 4 is an explosive view of the destructible barrel 3 shown in FIG. 3. The needle seat 34 passes through the opening 46 of the destruction device 38 and the opening part 42 of the cover 40. And the needle seat 34 supports the needle hub 16. The injection fluid contained in the body 32 flows through the opening 48 of the needle seat 34 to the needle hub 16, and then is injected into the target (not shown). The destruction device 38, such as a knob, is provided to break the connection portion 36 (shown in FIG. 3) between the body 32 and the needle seat 34.

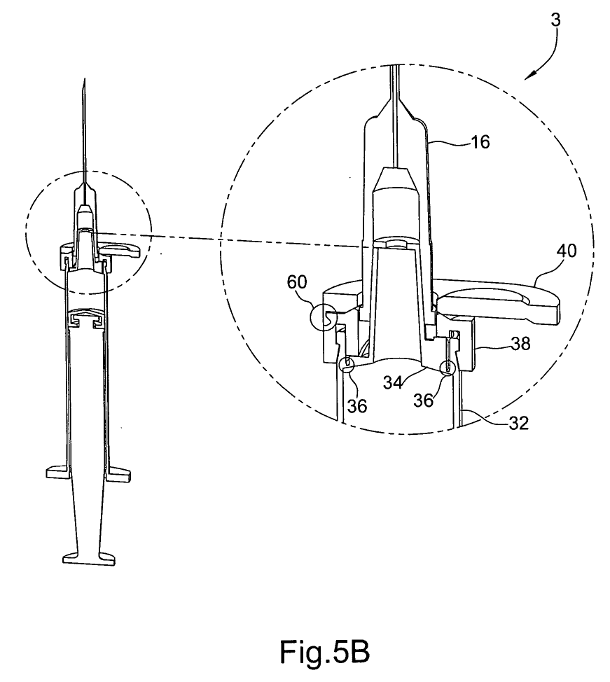

FIG. 5A and FIG. 5B further illustrate the structure of the destructible barrel 3 and the relation among the body 32, the needle seat 34, and the destruction device 38. The first end 321 of the body 32 has a first lock portion 52, and the destruction device 38 has a second lock portion 54. The second lock portion 54 of the destruction device 38 is rotatably connected to the first lock portion 52 of the body 32. The first lock portion 52 and the second lock portion 54 can be any well-known components, such as a clasp or a latch. The needle seat 34 has a first spiral surface 56, and the destruction device 38 correspondingly has a second spiral surface 58. Although three sets of spiral surfaces are illustrated in FIG. 5A as an exemplary embodiment, the first spiral surface 56 and the second spiral surface 58 can respectively be one or more sets of surfaces in practice. The cover 40 is movably connected to the destruction device 38 by any well-known connection, such as a clasp shown in the region 60 in FIG. 5B.

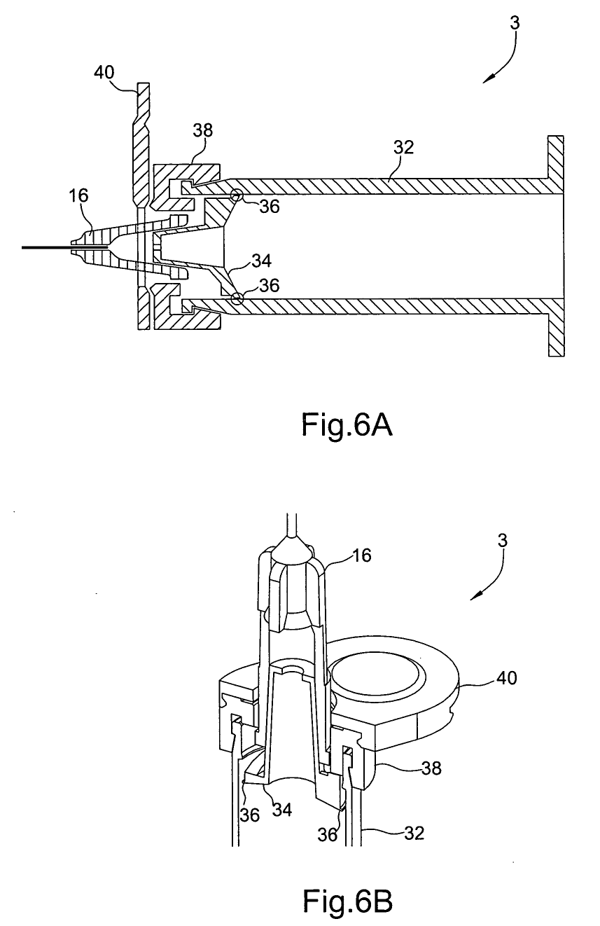

FIG. 6A and FIG. 6B illustrate the needle seat 34 destructively separated from the body 32 by the operation of the destruction device 38. After use of the destructible barrel 3, breaking the destructible barrel 3 can prevent inappropriate recycling and reuse of the destructible barrel 3. Because there is a height difference between the first spiral surface 56 and the second spiral surface 58 shown in FIGS. 5A and 5B, rotating the destruction device 38 will make the second spiral surface 58 push against the first spiral surface 56 of the needle seat 34 along the axis of the destructible barrel 3. Since the strength of the connection portion 36 is weaker than the strength of the needle seat 34, the connection portion 36 is broken when the destruction device 38 pushes the needle seat 34. Thus, the needle seat 34 is destructively separated from the body 32. With the destruction device 38, the destructible barrel 3 can be broken by a simple operation and can not be reused.

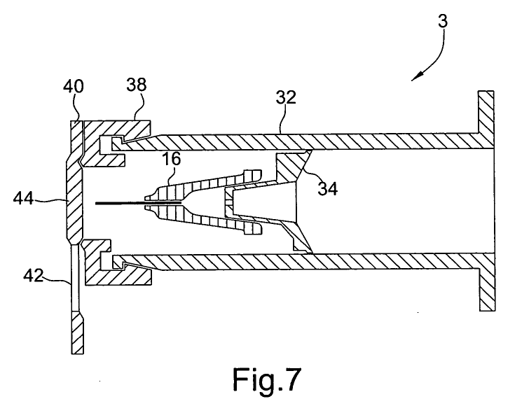

As shown in FIG. 7, after the needle seat 34 is separated from the body 32, the cover 40 is further moved to shelter the first end 321 of the body 32 with the closed part 44 of the cover 40. The cover 40 can be moved by any known method, such as sliding the cover 40 along a track (not shown). The cover 40 includes an opening part 42 and a closed part 44 in this embodiment, but one skilled in the art can easily make some modifications to the cover 40 with similar functions. For example, a cover (not shown) without an additional opening part can be rotatably disposed on the first end 321 of the body 32, and can be rotated to shelter the first end 321. The destructible barrel 3 sheltered by the cover 40 can be easily recognized as a used barrel. Furthermore, the used needle hub 16 and the needle seat 34 can be accommodated inside the body 32 of the barrel 3 and blocked by the cover 40, so that they will not cause accidental injuries.

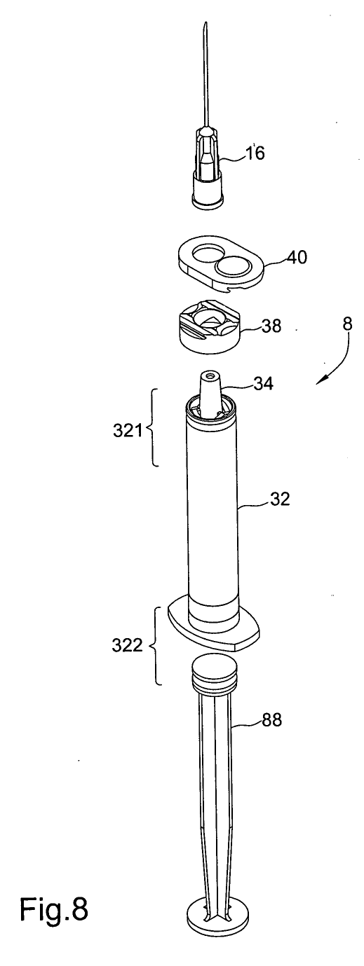

FIG. 8 is an explosive view of a destructible safety syringe 8 in accordance with another embodiment of the present invention. The syringe 8 includes a needle hub 16, a body 32, a needle seat 34, a destruction device 38, a cover 40, and a plunger 88. The body 32 contains an injection fluid (not illustrated). The body 32 has a first end 321 and a second end 322. The needle seat 34 is connected to the body 32 through a connection portion 36. The plunger 88 is disposed inside the body 32 and is able to move along the axis to drive the injection fluid. The destruction device 38 is movably connected to the first end 321 of the body 32. The body 32 and the needle seat 34 are molded as an integral one-piece casting, and can be made by any well-known method, such as injection molding. Thus, not only the production method is simplified, but the production cost is lowered. Furthermore, an additional elastic ring between the body 32 and the needle seat 34 is not required since there is no fluid leakage problem. The cover 40 has an opening part 42 and a closed part 44. The cover 40 is movably connected to the destruction device 38. The opening part 42 exposes the first end 321 of the body 32, and the needle seat 34 passes the cover 40 through the opening part 42.

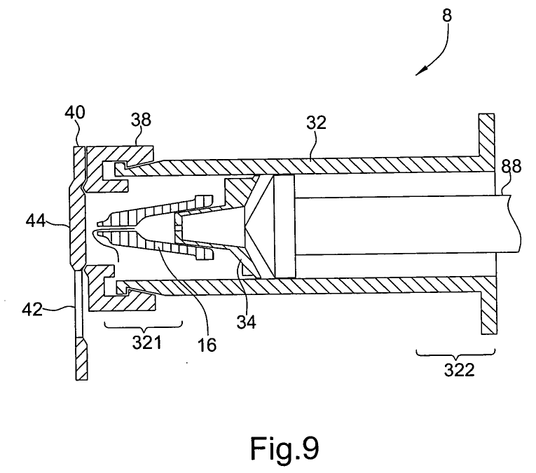

The breaking mechanism of the destruction device 38 of the embodiment of FIG. 8 is the same as the destructible barrel 3 of the previous embodiment, and is not elaborated here for conciseness. FIG. 9 further illustrates the breaking of the needle hub 16. After the needle seat 34 is separated from the body 32, the needle hub 16 and the needle seat 34 can be accommodated in the body 32 of the barrel 3. The cover 40 is pushed to shelter the first end 321 of the body 32. Thereafter, the plunger 88 is further pushed to compress the needle hub 16 and needle seat 34. The needle hub 16 is deformed and broken by the compressing force between the cover 40 and the plunger 88. The syringe 8 can not be reused since both the needle hub 16 and the needle seat 34 are broken. Furthermore, the syringe 8 sheltered by the cover 40 can be easily recognized as a used barrel.

The above description is intended to set forth exemplary embodiments of the invention, and is not intended to limit the scope of the invention in any way. Rather, various changes may be made in the function and configuration of the elements described in the embodiment without departing from the spirit and scope of the invention. Thus, the protected scope of the present invention is as set forth in the appended claims.

What is claimed is:

1. A destructible barrel used in a syringe, comprising:
a body for containing an injection fluid, the body having a first end and a second end;
a needle seat connected to the first end of the body through a connection portion; and
a destruction device being movably engaged with the first end of the body;
wherein, responsive to an user operation, the destruction device breaks the connection portion to destructively separate the needle seat from the body.

2. The destructible barrel of claim 1, wherein the needle seat and the body are molded as an integral one-piece casting.

3. The destructible barrel of claim 1, wherein the destruction device includes a knob being rotatably engaged with the first end of the body.

4. The destructible barrel of claim 3, wherein the needle seat further comprises a first spiral surface and the knob further comprises a second spiral surface corresponding to the first spiral surface.

5. The destructible barrel of claim 4, wherein the user operation comprises rotating the knob to make the second spiral surface push against the first spiral surface, and the connection portion is broken consequently to separate the needle seat from the body.

6. The destructible barrel of claim 3, wherein the needle seat further comprises a plurality of first spiral surfaces, and the knob further comprises a plurality of second spiral surfaces corresponding to the plurality of the first spiral surfaces, and wherein the user operation comprises rotating the knob to make the plurality of the second spiral surface push against the plurality of the first spiral surface, and the connection portion is broken consequently to separate the needle seat from the body.

7. The destructible barrel of claim 1, further comprising a cover disposed on the first end of the body, the cover being pushed to shelter the first end of the body after the needle seat is separated from the body.

8. The destructible barrel of claim 7, wherein the cover further comprises an opening part and a closed part, and the opening part exposes the first end of the body before the needle seat is separated from the body, and the cover is pushed allowing the closed part to shelter the first end of the body after the needle seat is separated from the body.

9. A destructible safety syringe, comprising:
a needle hub;
a barrel including a body, a needle seat and a destruction device, the body containing an injection fluid, and the body having a first end and a second end, the needle seat being connected to the first end of the body through a connection portion, and the needle seat supporting the needle hub, the destruction device being movably engaged with the first end of the body; and
a plunger being disposed inside the body and being able to move along the axis; wherein, in response to an user operation, the destruction device breaks the connection portion to destructively separate the needle seat from the body.

10. The destructible safety syringe of claim 9, wherein the needle seat and the body are molded as an integral one-piece casting.

11. The destructible safety syringe of claim 9, wherein the destruction device includes a knob being rotatably engaged with the first end of the body.

12. The destructible safety syringe of claim 11, wherein the needle seat further comprises a first spiral surface and the knob further comprises a second spiral surface corresponding to the first spiral surface.

13. The destructible safety syringe of claim 12, wherein the user operation comprises rotating the knob to make the second spiral surface push against the first spiral surface, and the connection portion is broken consequently to separate the needle seat from the body.

14. The destructible safety syringe of claim 11, wherein the needle seat further comprises a plurality of first spiral surfaces, and the knob further comprises a plurality of second spiral surfaces corresponding to the plurality of the first spiral surfaces, and wherein the user operation comprises rotating the knob to make the plurality of the second spiral surface push against the plurality of the first spiral surface, and the connection portion is broken consequently to separate the needle seat from the body.

15. The destructible safety syringe of claim 9, further comprising a cover disposed on the first end of the body, the cover being pushed to shelter the first end of the body after the needle seat is separated from the body.

16. The destructible safety syringe of claim 15, wherein the cover further comprises an opening part and a closed part, and the opening part exposes the first end of the body before the needle seat is separated from the body, and the cover is pushed allowing the closed part to shelter the first end of the body with the closed part after the needle seat is separated from the body.

17. The destructible safety syringe of claim 15, wherein when the cover shelters the first end of the body, the needle seat is pushed by the plunger to break the needle hub.
